(19) United States
(12) Patent Application Publication
(10) Pub. No.: US 2007/0021838 A1
(43) Pub. Date: Jan. 25, 2007

(54) Site specific minimally invasive joint implants

(76) Inventors: Jeffrey R. Dugas, Vestavia Hills, AL (US); Edward Lyle Cain JR., Mountain Brook, AL (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/186,911

(22) Filed: Jul. 22, 2005

(51) Int. Cl. A61F 2/46; A61F 2/38; A61B 17/90; A61B 17/80

(52) U.S. Cl. 623/20.3; 623/908; 606/88; 606/69

(57) ABSTRACT

A prosthetic articular surface for implantation in a joint such as the knee is provided. The prosthetic articular surface comprises a contoured plate having two sides, one side of which is a bone-contacting side and the other side of which is an articular side, and at least one post extending from the bone contacting side. A method of placing the prosthesis at an implant site, a guide tool for facilitating implantation, and a surgical kit containing the prosthesis also are provided. The prosthetic articular surface, method of implanting the prosthesis, guide tool for facilitating implantation, and surgical kit containing the prosthesis may be used to replace or augment a damaged joint in the knee.

### FIELD OF THE INVENTION

Embodiments of the invention relate to joint implants and methods of placing the joint implants. More particularly, embodiments relate to prosthetic articular surfaces for replacement and augmentation of articulating surfaces of the join, such as the knee, methods of implanting the prostheses, and surgical kits that facilitate successful surgical placement of the prostheses.

### BACKGROUND

The knee joint is a complex articulating structure linking the tibia and femur of the leg. The knee has been characterized as comprising three separate articulating joints (Henry Gray, F. R. S., Gray's Anatomy, 274 (1974 Running Press)). Two of these joints are condyloid joints located between each condyle (the oval-like head) of the femur and the corresponding tuberosities (the elliptical sockets) of the tibia. The third joint is a semi-arthrodial joint between the patella and the femur.

Like all articulating joints, knee joints are subject to degradation and damage due to, for example, disease, acute injury, and chronic wear. In particular, the articular cartilage that is found at the articulating surfaces of the knee joint can be damaged. Typically, articular cartilage comprises a significant amount of hyalin cartilage, the healing of which depends upon bleeding from the bone at the damaged joint. However, bleeding at damaged articulating knee joints often is limited, resulting in incomplete or inadequate repair of damaged cartilage. Additionally, damaged hyaline cartilage, if healed, often is replaced with less-durable fibrocartilage.

Therefore, various procedures have been proposed and practiced in order to repair and augment damaged knee joints. One such procedure, abrasion arthroplasty, comprises abrading the surface of the bones at the damaged joint in order to stimulate bleeding and subsequent healing of the damaged joint. The microfracture technique, which also induces bleeding, does so by exposing bone and then creating microfractures in the bone. Additionally, various medications delivered both orally and parenterally (e.g. by injection at the knee) have been suggested. Cartilage transplants to replace or augment the damaged joint also have been proposed. Perhaps the most radical of the procedures is the total knee replacement (TKR) surgery, wherein the entire cartilage surface is removed and replaced with a prosthetic substitute.

The description herein of problems and disadvantages of known apparatuses, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

Various embodiments described herein provide an improved method of repairing damaged articular surfaces of the knee and other joints. Further, such embodiments provide an improved implantable device or prosthesis that can replace and augment damaged articular surfaces and articular cartilage. Additionally, various embodiments provide an improved surgical kit to facilitate rapid and successful implantation of prosthetic articular surfaces for replacement and augmentation of articular surfaces of the knee and other joints. Furthermore, various embodiments provide an improved guide tool to facilitate the removal of cartilage and subchondral bone in the joint (e.g., the knee) at the implantation site of a prosthetic articular surface. Also, various embodiments provide an improved method of implanting a prosthetic articular surface in the knee and other joints.

Therefore, in accordance with an embodiment of the invention, there is provided a method for implanting a prosthetic articular surface in a joint (e.g., the knee). The method comprises identifying a site on an articular surface of a joint where the prosthesis is to be implanted; selecting a proper prosthesis for implantation at the site from a plurality of shapes in a kit; and placing the prosthesis at the site. To achieve this, cartilage and subchondral bone may be removed, such as by reaming or other removal techniques.

Embodiments also provide a prosthetic articular surface for implantation in a knee. The prosthesis comprises a contoured plate having two sides, one side of which is a bone-contacting side and the other side of which is an articular side. The bone-facing side may be secured by bone cement, for example. Additionally, the prosthesis comprises at least one post extending from the bone contacting side. The cross-section of the prosthesis may be selected from a circle, oval, └-shape, ┘-shape, and triangular shape.

Embodiments further provide a guide tool to facilitate implantation of the prosthesis. The guide tool comprises a template with a shape and size substantially similar to the cross-sectional geometry and size of the prosthesis, and a top surface with a radius of curvature substantially similar to the radius of curvature of the prosthesis.

Embodiments additionally provide a surgical kit to facilitate rapid and successful implantation of prosthetic articular surfaces. The surgical kit comprises one or more prosthetic articular surfaces as described herein. The kit optionally further comprises a guide tool as described herein, a reamer bit, and a drill bit. Preferably, the surgical kit comprises several prosthetic articular surfaces with varying cross-sectional geometries, radii of curvature, and sizes. Also, the surgical kit preferably comprises a guide tool corresponding to each of the prosthetic articular surfaces included in the kit.

While the invention will be described herein with particular reference to a knee joint, it is to be appreciated that the invention also may be used in reference to other articular joints in the body in need of replacement or augmentation, such as hip and shoulder joints. One skilled in the art therefore will appreciate other corresponding applications of the invention, in accordance with the guidelines herein.

These and other features and advantages of the embodiments will be apparent from the description provided herein.

### BRIEF DESCRIPTION OF THE DRAWINGS

FIG. 1, embodiments A and B, is a drawing of exemplary prosthetic articular surfaces implanted in, respectively, a femur and tibia.

FIG. 2, embodiments A-J, is a drawing of exemplary prosthetic articular surfaces having various cross-sectional geometries.

FIG. 3, embodiments A-E, is a drawing of an exemplary prosthetic articular surface and corresponding guide tool.

FIG. 4 is a drawing of an exemplary reamer and use thereof.

### DETAILED DESCRIPTION OF THE EMBODIMENTS

The following description is intended to convey a thorough understanding of the various embodiments of the invention by providing a number of specific embodiments and details involving devices and methods of augmenting and repairing damaged articular surfaces and cartilage in a knee joint. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

Throughout this description, the term “cross-sectional geometry,” in reference to the prosthetic articular surface's “cross-sectional geometry,” is intended to mean the two-dimensional geometry, or shape, of the prosthesis when viewed from a plan view. A plan view of the prosthesis is the two-dimensional view of the prosthesis taken from the articular side of the plate comprising the prosthesis.

Embodiments of the invention provide a method of implanting a prosthetic articular surface in a knee joint. The prosthetic articular surface may repair and augment damaged, injured, and diseased natural articular surfaces and cartilage of the knee joint. For example, the prosthetic articular surface may be used to replace the damaged natural articular surface and cartilage. The method comprises identifying a site on an articular surface of a joint where the prosthesis is to be implanted; selecting a proper prosthesis for implantation at the site from a plurality of shapes in a kit; optionally reaming out a volume of cartilage and subchondral bone at the site where the prosthesis is to be implanted; and placing the prosthesis at the site. Also, a guide tool optionally may be selected corresponding to the prosthesis and placed at the site where the prosthesis is to be implanted. The guide tool may be used during reaming out of the bone preparatory to placing the prosthesis. Furthermore, a lug hole may be drilled at the site in order to accept the prosthesis.

In one embodiment, the method may be carried out using minimally invasive surgical techniques. Particularly, the acts of identifying the site, placing the guide tool, reaming out the volume of bone, drilling the lug hole, and placing the implant each may be accomplished using minimally invasive surgical techniques. One skilled in the art of surgery will appreciate how these surgical techniques can be used in this context. For example, endoscopes, micro-sized reamer bits and drill bits, flexible and semi-flexible implants and guide tools, and so forth may be used in accordance with minimally invasive surgical techniques.

Generally, in the case of use of this implant for the knee, the site where the prosthetic articular surface is to be implanted is an articular surface including, but not limited to, the lateral femoral condral (LFC) surfaces, medial femoral condral (MFC) surfaces, trochlea surfaces, patella surfaces, tibia surfaces (e.g. surfaces of the tuberosities of the tibia), and combinations and portions thereof. For example, implants may be placed on portions of these surfaces, over one or more entire surfaces, and on one or more surfaces of the same knee. Where the prostheses are to be sited depends upon the clinical needs of the implant recipient as determined by the surgeon.

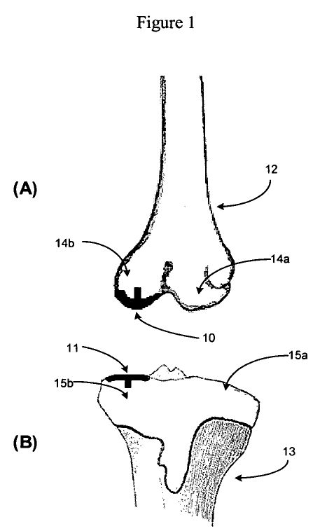

FIG. 1, embodiments A and B, illustrates the exemplary placement of prosthetic articular surfaces according to embodiments of the invention at, respectively, a femoral condral surface in a femur and a tuberosity surface in a tibia. In FIG. 1, a femur 12 (embodiment A) and tibia 13 (embodiment B) are depicted. The femur has condyle 14a and 14b, one of which has been augmented with a prosthetic articular surface 10. The tibia has tuberosities 15a and 15b, one of which has been augmented with a prosthetic articular surface 11.

The site at which a prosthetic articular surface is to be implanted can be selected by one of skill in the art, for example, on the basis of inflammation and observed damage of the articular surfaces of the knees. Procedures such as diagnostic and surgical arthroscopy (e.g. stereoscopy) and fluoroscopy (including X-ray, CT scan, C-arm fluoroscopy, and other techniques) may be utilized in order to determine what surfaces of the knee joint may benefit from augmentation or replacement using the prosthetic articular surfaces provided by the embodiments.

After a site for treatment has been identified, an appropriate prosthetic articular surface may be selected for placement (i.e. implantation) at the site. The embodiments provide prosthetic articular surfaces comprising a contoured plate having two sides, one side of which is a bone-contacting side, meaning that it contacts the bone into which the prosthesis is to be implanted. The opposite side of the plate is an articular side, meaning that it is to act as a replacement or augmentation of the articular surface that has been removed for placement of the prosthesis. Additionally, the prosthesis may comprise at least one post extending from the bone contacting surface. The cross-section of the prostheses may be selected from a circle, oval, └-shape, ┘-shape, triangular shape, and so forth, so that an appropriately shaped prosthesis based upon the needs of the treatment site may be selected. The prostheses provided by the embodiments also may have a variety of different sizes, so that an appropriately sized prosthesis for the treatment site may be selected. Additionally, the radius of curvature of the prostheses, in part a function of the contouring of the plate, may vary so that an appropriately curved prosthesis for the treatment site may be selected. In general, the varying geometry, size, and radius of curvature of the prosthetic articular surfaces provided by the embodiments allow for a prosthesis particularly suited to a given treatment site to be selected and placed at the treatment site during the implantation surgery, thereby avoiding the need for multiple surgeries (e.g. one implant the prosthesis). In one embodiment, the implant may be secured directed to the bone, such as through the use of bone cement or other adhesives or connectors.

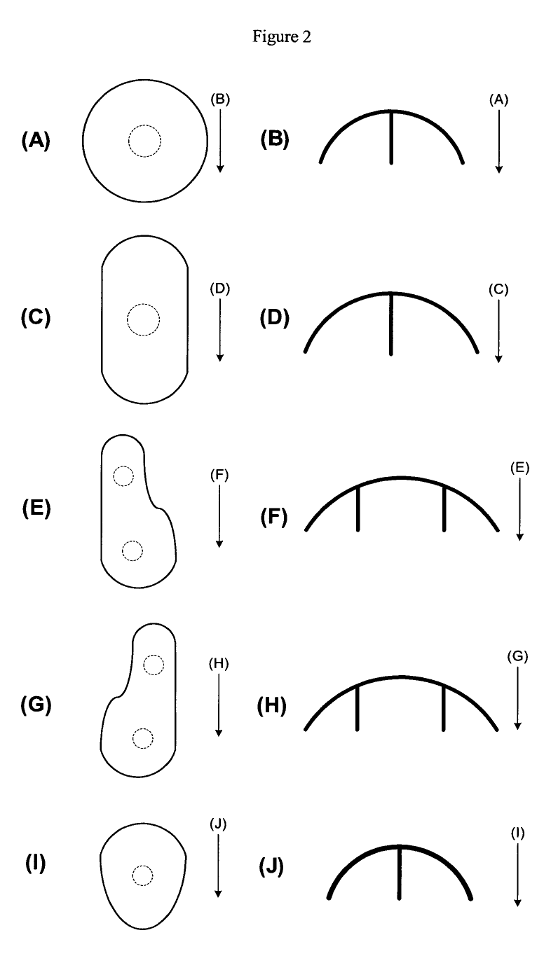

FIG. 2, embodiments A-J, illustrates several exemplary prosthetic articular surfaces according to embodiments of the invention. Embodiments A, C, E, G, and I provide plan views of the prostheses, from which their cross-sectional geometries may be examined. In embodiments A and B, a circular prosthesis with a single post extending therefrom is shown. In embodiments C and D, an oval or elliptical prosthesis with a single post extending therefrom is shown. In embodiments E and F, a └-shaped prosthesis with two posts extending therefrom is shown. In embodiments G and H, a ┘-shaped prosthesis is shown with two posts extending therefrom. In embodiments I and J, a triangular shaped prosthesis is shown with a single post extending therefrom. An appropriately shaped prosthesis from among those depicted in FIG. 2, and other such applicable geometries and shapes, may be selected in accordance with the site of implantation. For example, a triangularly shaped prosthesis may be most appropriate for implantation at the intercondyloid notch, a circular shaped prosthesis may be most appropriate for implantation at a tuberosity in the tibia, and a └-shaped or ┘-shaped prosthesis may be most appropriate for implantation at a lateral or medial condyl in the femur.

The post(s) on the bone-contacting side of the prosthesis may include additional elements to aid in securing the post to the lug hole that may be drilled for placement of the prosthesis. For example, a plurality of barbs, threads, ribs, fins, milled slots, tapered distal features, features to prevent rotational movement of the prosthesis, and features to increase friction between the prosthesis and the lug hole may be included on the post(s) extending from the bone-contacting side of the prosthesis. These additional feature preferably aid in securing the prosthetic articular implant to the treatment site by restricting or preventing separation of the post(s) from the lug holes in which they are to be placed during implantation of the prosthesis.

In order to place the prosthesis at the treatment site, it may be desirable to remove a volume of cartilage and subchondral bone. To accomplish this, a guide tool may be used in conjunction with a drill equipped with an appropriate reamer bit in order to remove the volume of cartilage and subchondral bone necessary for proper placement of the prosthesis. Sufficient bone may be removed such that the edge of the articular side of the prosthetic articular surface, when implanted, is approximately flush with the remaining natural cartilage surrounding the site where implantation occurs. Generally, cartilage and subchondral bone may be removed to a depth equal to the thickness of the prosthesis. For example, as can be seen in FIG. 1, embodiments A and B, the edges of the articular sides of the prosthetic articular surfaces 10 and 11 are approximately flush with the surface of the femur 12 and tibia 13, respectively, in which they are transplanted. This may be desirable so that there is a seamless and smooth transition from the natural cartilage to the prosthetic articular surface so that the prosthetic articular surface functions substantially similar to the natural articular surface it replaces and/or augments. The volume of bone that is to be removed for placement of the prosthetic articular surface therefore will depend upon the size and shape of the prosthesis, as well as the shape of the articular bone surface into which the prosthesis is to be implanted.

Embodiments of the invention provide guide tools to aid in removal of a volume of cartilage and subchondral bone for placement of a prosthetic articular surface. The guide tool may comprise a template (i.e. a ring or mask). The template may have an inner surface, an outer surface, and a top and bottom. The template may comprise a circular shape, as well as many other shapes. The template may be shaped substantially similar to a given prosthetic articular surface to which it corresponds. Additionally, the template may be substantially similar in size, or slightly oversized, compared to the prosthetic articular surface to which it corresponds. Finally, the radius of curvature of the top surface of the template may be substantially similar to the radius of curvature of the prosthetic articular surface to which it corresponds. In this way, the guide tool may act as a template (similar to a cookie cutter) or mask to guide removal of bone in preparatory to implantation of the prosthetic articular surface to which the guide tool corresponds.

The guide tool may be placed at the previously identified implantation or treatment site so that the tool defines a surface area of the bone within its confines (i.e. within the area defined by the inner surface of the guide tool). Then, a drill equipped with a reamer bit may be used to remove bone within the confines of the guide tool. Removal of cartilage and subchondral bone may be restricted to the surface area of the cartilage and subchondral bone within the outline of the guide tool. Use of the guide tool is advantageous because free-hand removal of bone before implantation of the prosthetic articular surface generally is more difficult and less precise than removal using the guide tool. Additionally, use of the guide tool may speed the process of cartilage and subchondral bone removal and result in a faster and more successful surgical placement of the prosthesis.

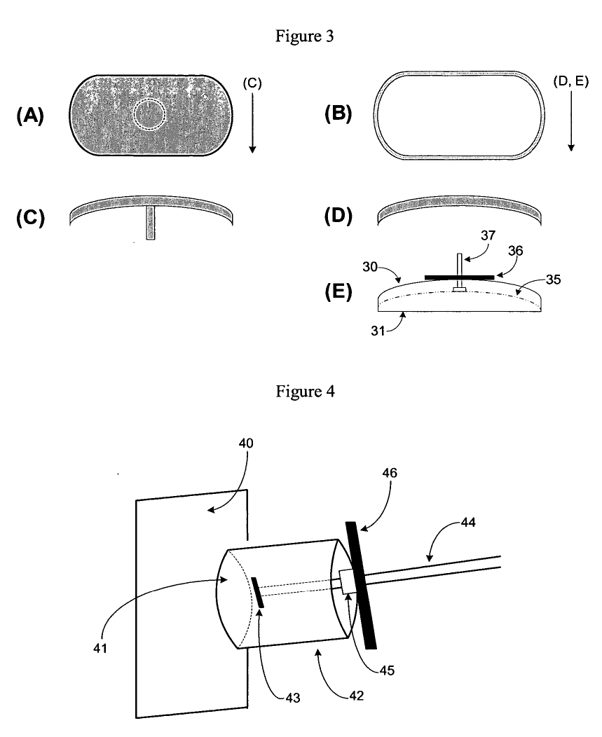

FIG. 3 depicts an exemplary prosthetic articular surface and a corresponding guide tool. In embodiments A and C, an exemplary prosthetic articular surface is shown. Embodiments B, D, and E show two versions of a corresponding guide tool for use in excising a volume of cartilage and subchondral bone for placement of the prosthetic articular surface shown in embodiments A and C. As seen in embodiment B, the guide tool comprises a template (or mask or ring) in the shape of the prosthesis. The guide tool may be substantially the same size, or slightly larger, than the prosthesis to which it corresponds.

As seen in embodiments C (prosthesis) and D (guide tool), the guide tool also may have the same radius of curvature at the prosthesis. Also, as seen in embodiment E (guide tool), the guide tool may have a top surface 30 with a radius of curvature substantially similar to that of the prosthesis, but a bottom surface 31 with a different radius of curvature. However, when used in conjunction with a depth gauge, the guide tool in embodiment E still may be appropriate for directing the removal of cartilage and subchondral bone for placement of the corresponding prosthesis. As seen in E, the depth gauge may comprise, in part, a sleeve 36 attached to a reamer bit 37 that limits the depth to which the reamer bit may extend. The sleeve 36 is intended to glide or be slidably displaced along the top surface 30 of the guide tool. The reamer bit 37 therefore may excise cartilage and subchondral bone to a depth indicated by line 35, which has the same radius of curvature as the top surface 30 of the guide tool, which in turn has a radius of curvature substantially similar to that of the prosthesis to which the guide tool corresponds. Therefore, it is desirable that the top surface of the guide tool, on which the depth gauge is intended to rest, is substantially the same curvature as the prosthesis.

The guide tool may provide a fast and accurate method of removing bone in the pattern of the prosthetic articular surface that is to be implanted. The guide tool may be produced from a flexible material such as a polymeric material so that the tool can be deformed and placed at the implantation site in a minimally invasive fashion. For example, if the guide tool is sufficiently flexible, it can be inserted into the knee using a catheter and then expand therein to reach its full size, in which state it may function as a template or mask to direct where bone removal is to occur.

Embodiments of the invention also provide a depth gauge that can be used in conjunction with the guide tool and a reamer bit. The depth gauge may direct the depth to which cartilage and subchondral bone is removed within the portion of the surface defined by the guide tool. This practice is designed to match the edge of the articular side of the prosthetic articular surface, as closely as possible, with the height of the cartilage surrounding the implantation site.

The depth gauge may comprise a collar that releasably engages the shaft of a reamer bit. The collar of the depth gauge therefore may be loosened and tightened in order to move up and down the shaft of the reamer bit and be secured at a certain position (i.e. height) on the shaft. The collar may be capable of being slidably displaced along the inside surface of the guide tool so that the depth gauge can trace the outline of the guide tool. A sleeve may be connected to the collar of the depth gauge. The sleeve may be capable of contacting the top surface of the guide tool (i.e. the ring, mask, or template). Additionally, the sleeve may be shaped and sized in a manner such that it cannot fit within the confines of the guide tool. In this manner, the sleeve, in combination with the collar (the two of which comprise the depth gauge) and the guide tool, may prevent the reamer bit from penetrating beyond a certain depth into the bone. The shaft of the reamer bit may be marked with indicia corresponding to the depth to which the bit may penetrate for a given position of the collar in reference to the reamer bit.

An exemplary depth gauge is depicted in FIG. 4. In FIG. 4, plane 40 signifies a joint surface. A guide tool 42 has been placed on the surface, thereby defining a portion of the cartilage and subchondral bone 41 which is to be reamed out using the reamer bit 43. To the shaft 44 of the reamer bit a collar 45 is releasably attached. The collar may be loosened and tightened so as to releasably engage the shaft of the reamer bit in order to be slidably disposed along the shaft. The collar preferably is slidably disposable about the interior surface of the guide tool 42. A sleeve 46 is attached to the collar. The sleeve, by virtue of its shape and/or size, cannot enter within the confines of the guide tool 42, and therefore instead is limited to sliding along the top surface of the guide tool. In this manner, the depth gauge (comprising the collar and sleeve) may limit and/or prevent the reamer bit from extending beyond a certain depth into the bone.

The guide tool and depth gauge may be used to ream out the outline of the prosthetic articular surface and to ream out the remaining cartilage and subchondral bone in the center of the volume where the prosthesis is to be placed. It also may be desired to ream out the area of cartilage and subchondral bone to increasing depths in a stepwise manner. If the volume of cartilage and subchondral bone is reamed out in a stepwise manner, the placement of the prosthesis may be examined at each depth and, if the elevation of the prosthesis above the cartilage's surface is found to be excessive, the depth may be increased until, for example, the edge of the articular side of the prosthesis lies flush with the surface of the joint or some other proper placement of the prosthesis occurs. During removal of a volume of cartilage and subchondral bone, irrigation using water or saline solution and vacuum may be desired to remove excised bone shards and fragments from the implantation site.

Before the prosthetic articular surface may be placed at the implant site, a lug hole may be drilled to accept the post extending from the bone-contacting side of the prosthesis. The lug hole can be drilled simply by using an appropriate drill bit. To place the prosthesis, bone cement or some other appropriate adhesive may be used in order to secure the prosthesis in place. Also, the prosthesis may be secured by press-fitting the prosthesis into the bone. In this case, it may be desirable to slightly undersize the lug hole so that the prosthesis is firmly held in place by the post in the slightly undersized lug hole. Also, pins may be used to secure the prosthesis to the implant site. Still other methods of securing the prosthesis in place at the implant site will be appreciated by one of skill in the art, and all such methods, alone or in combination, in accordance with the guidelines herein are contemplated for use in the invention.

Embodiments of the invention also provide surgical kits comprising a plethora of prosthetic articular surfaces from which an orthopedic surgeon may select an appropriate prosthesis for implantation at the designated site(s), for example sites in the knee.

The surgical kits provided by the embodiments comprises at least one articular prosthetic surface as has been described herein. The articular surface may be selected from those having a cross-sectional geometry of a circle, an oval, a └-shape, a ┘-shape, and a triangle. In one embodiment, the surgical kit comprises at least one prosthesis for each of these geometries. Also, for each of these geometries the kit may comprise prostheses having a variety of sizes. In one specific embodiment, for each of the sizes of each of the geometries, the kit comprises prostheses having a variety of different radii of curvature. Therefore, the kit may provide a wide range of geometries, sizes, and radii of curvature from which to choose when selecting a prosthetic articular surface for implantation. In this way, a surgeon may choose a prosthesis that is most suited for implantation at the designated treatment site from a single surgical kit during the operation.

The surgical kit may additionally comprises a reamer bit that may be used to remove a volume of cartilage and subchondral bone where the prosthesis is to be placed. Also, the surgical kit may comprise one or more guide tools as has been described herein for use in directing the removal of cartilage and subchondral bone for implantation of the prosthesis. The surgical kit may comprise a guide tool corresponding to each of the prosthetic articular surfaces found in the kit. In other words, the surgical kit may comprise a guide tool available for use with each unique prosthetic articular surface (on the basis of geometry, size, and radius of curvature) in the kit.

The prosthetic articular surfaces provided by the embodiments may be made of any appropriate biocompatible material. For example, oxinium available from Smith and Nephew, Memphis, Tenn., is a one material for the prosthetic articular surfaces. Oxinium is a zirconium or zirconium-alloy based material coated with zirconium oxide or zirconium nitride, as discussed in U.S. Pat. No. 5,370,694, the disclosure of which is incorporated by reference herein in its entirety.

Other materials appropriate for fabrication of the prosthetic articular surfaces include medical alloys such as titanium and titanium alloys (e.g. Ti-6A1-4V), tantalum and tantalum alloys, stainless steel alloys (e.g. 316 L), cobalt-based alloys, cobalt-chromium alloys, cobalt-chromium-molybdenum alloys, cobalt-chromium-tungsten-nickel alloys, chromium-nickel-manganese alloys, niobium alloys, zirconium alloys, nickel and nickel alloys; medical plastics such as polyvinyl chlorides, polypropylenes, polystyrenes, acetal copolymers, polyphenyl sulfones, polycarbonates, acrylics, silicone polymers, polyetheretherketone (PEEK), polyurethanes, polyethylenes, polyethylene terphalate (PET), polymethylmethacrylate (PMMA), and polycaprolactone; ceramics such as alumina, zirconia, hydroxyapatite, and calcium phosphate; and mixtures and combinations thereof. Also, natural substrates such as allograft, xenograft, and autograft bone may be used to fabricate the prosthetic articular surfaces. Prosthetic articular surfaces according to the embodiments also may be composites of one or more medical plastics, metals, alloys, ceramics, and bone (e.g. allograft, xenograft, and autograft bone).

In one embodiment, the prosthetic articular surfaces additionally may comprise various surface modifications in order to increase the prostheses' ability to function as a replacement or augmentation for an articular surface of the knee joint. The bone-contacting portion of the prosthesis (including the bone-contacting side of the plate and the post extending therefrom) may be treated to impart improved osteoinductive and/or osteoconductive properties. Additionally, the articular side of the prosthesis may be treated in a manner to increase its durability and performance as an articulating surface.

Exemplary treatments of the bone contacting portion of the prosthesis include surface treatments to encourage osteoinduction and osteoconduction. For example, biological activity such as bone in-growth and on-growth may be promoted by the creation of nano-scale surface features on the bone contacting portion of the prosthetic articular surface. It is thought that nano-scale surface features, because of their size, may have advantageous interactions with proteins on the surfaces of adjacent bone cells, thereby promoting in-growth and on-growth of bone. Additionally, general surface roughening and the creation of appropriately sized pores is thought to have osteoconductive and osteoinductive effects. Physical surface modification may be affected by the use of known processes such as machining, grinding, grit blasting, chemical etching, chemical vapor deposition, physical vapor deposition, electric discharge processes, laser etching, and the application of textured surfaces (e.g., textured cladding secured by welding, bonding, mechanical fixation, etc.).

In another embodiment, osteoinductive and osteoconductive solutions, materials, and so forth may be incorporated into or on the surface of the bone contacting portion of the prosthetic articular surface. For example, osteoinductive and osteoconduction materials may be deposited as a layer on part or all of the bone-contacting portion of the prosthesis. Also, osteoinductive and osteoconductive materials may be incorporated into the material itself (e.g. as an impurity) that comprises the prosthesis. Also, osteoinductive and osteoconductive materials may be adsorbed onto the surface of the bone-contacting portion of the prosthesis. One skilled in the art will appreciate how these materials may be applied to the prosthesis, in accordance with the guidelines herein.

Known osteoinductive and osteoconductive materials include, but are not limited to, growth factors such as endothelial cell growth factor (ECGF), insulin-like growth factor (IGF-1), platelet derived growth factors (PDGF), epidermal growth factor (EGF), and fibroblast growth factors (FGF, bFGF, etc.); hormones such as human growth hormone (HGH); mineral-based materials such as hydroxyapatite (HA) and tricalcium phosphate (TCP); and other substances such as cementum attachment extracts (CAE), ketanserin, collagen, fibronectin (FN), and osteonectin (ON). One skilled in the art will recognize other osteoinductive and osteoconductive solutions, materials, and so forth that may be used in accordance with the guidelines herein, and all such are contemplated for use in the embodiments.

Various treatments of the articular portion of the prosthetic articular surface are contemplated for use in the embodiments. Generally, treatments that result in a more durable surface are provided for use herein, as are treatments that decrease the likelihood of ejection of particles from the articular surface. For example, treatments to harden the articular surface such as, in the case of a metallic prosthetic articular surface, annealing, heat treatment (i.e. case hardening), and precipitation hardening may be used. In the case of a polymeric-based prosthetic articular surface, additionally cross-linking and the application of cladding or coatings to reinforce the articulating side of the prosthesis are applicable methods of treating the surface.

Additionally, the articulating side of the prosthesis may be benefited by treatments and applications that decrease its coefficient of friction, thereby increasing its utility an articulating surface. For example, polishing of the surface in the case of a metallic prosthesis is a treatment than can decrease the coefficient of friction of the articular side of the prosthesis.

Various advantages in the treatment of damaged and diseased articular surfaces of the knee are presented by the various devices and methods provided by embodiments of the invention. The prosthetic articular surfaces may be used to replace and augment articular surfaces of joints, such as knee joints, that have been damaged, for example, by disease, acute injury, and chronic wear. The method of implanting the prosthesis, as described herein, provides a single surgical procedure in which an appropriate prosthesis may be selected and placed at a treatment site. This decreases the extent of patient discomfort inherent in multiple surgeries to select and then implant a prosthesis.

The method is aided by the surgical kits, as provided by the embodiments and described herein, that give a surgeon a wide array of prostheses from which to choose when selecting one for implantation at a treatment site. The surgeon may chose a prosthesis on the basis of its shape, size, and radius of curvature. These variables may be selected either to mimic the natural articular surface that is to be replaced or augmented, or may be selected differently in order to produce a certain therapeutic effect. The surgical kits may be especially useful because the full requirements of the surgical site to be treated, and therefore the implant that is to be placed there, may not be known until the implantation site has been accessed by surgical means. Therefore, upon accessing the site, a surgeon may choose an appropriate implant directly from the kit, without the necessity of extensive pre-implantation surgery to examine the implantation site and fabrication of a customized implant for the patient. In other words, whereas two surgeries may be required to examine the implantation site to obtain measurements for the preparation of a customized implant for the patient, only one surgery is required using the surgical kits provided herein because the surgical kits already contain a plethora of implants from which an appropriate implant may be chosen at the time of implantation (i.e. during a single surgical procedure).

The foregoing detailed description is provided to describe the invention in detail, and is not intended to limit the invention. Those skilled in the art will appreciate that various modifications may be made to the invention without departing significantly from the spirit and scope thereof.

What is claimed is:

1. A method for implanting a prosthetic articular surface in a joint, comprising:
identifying a site on an articular surface of a joint where the prosthesis is to be implanted;
selecting a proper prosthesis for implantation at the site from a plurality of shapes in a kit; and
placing the prosthesis at the site.

2. The method of claim 1 further comprising the step of reaming out a volume of bone at the site where the prosthesis is to be implanted.

3. The method of claim 1, further comprising:
selecting a guide tool corresponding to the prosthesis; and
placing the guide tool at the site where the prosthesis is to be implanted;
reaming out a volume of bone at the site where the prosthesis is to be implanted using the guide tool.

4. The method of claim 1, further comprising drilling a lug hole at the site where the prosthesis is to be implanted.

5. The method of claim 1, wherein minimally invasive surgical techniques are used to identify the site, ream out the volume of bone, and place the prosthesis.

6. The method of claim 1, wherein the site is located at a knee joint.

7. The method of claim 6, wherein the site at a knee joint is a surface selected from the group of surfaces consisting of the lateral femoral condral (LFC) surfaces, medial femoral condral (MFC) surfaces, trochlea surfaces, patella surfaces, tibia surfaces, and combinations and portions thereof.

8. The method of claim 1, wherein selecting a proper prosthesis for implantation comprises selecting a prosthesis on the basis of its cross-sectional geometry, radius of curvature, and size.

9. The method of claim 1, wherein the prosthesis comprises a contoured plate having two sides, one side of which is a bone-contacting side and the other side of which is an articular side, and at least one post extending from the bone contacting side; wherein the cross-section of the contoured plate is selected from a circle, oval, └-shape, ┘-shape, and triangular shape.

10. The method of claim 2, wherein the guide tool comprises a template with a shape and size substantially similar to the cross-sectional geometry and size of the selected prosthesis, and a top surface with a radius of curvature substantially similar to the radius of curvature of the selected prosthesis.

11. The method of claim 2, wherein reaming out the volume of bone where the prosthesis is to be implanted comprises using a reamer bit to remove bone within an area of the bone's surface that is defined by placing the guide tool thereon.

12. The method of claim 11, wherein reaming out the volume of bone where the prosthesis is to be implanted comprises reaming out the outline of the prosthesis first, and then proceeding to ream out the center of the site where the prosthesis is to be implanted.

13. The method of claim 11, wherein reaming out the volume of bone where the prosthesis is to be implanted comprises reaming out the volume stepwise to increasing depths.

14. The method of claim 1, wherein reaming out the volume of bone where the prosthesis is to be implanted further comprises reaming out the volume to a depth such that the edge of the articular side of the prosthetic articular surface is approximately flush with the remaining natural bone surrounding the site.

15. The method of claim 1, wherein placing the prosthesis at the site further comprises cementing the prosthesis into the bone.

16. The method of claim 4, wherein placing the prosthesis at the site further comprises press-fitting the prosthesis into the bone.

17. A surgical kit for implantation of a prosthetic articular surface at a bone site in the knee, comprising one or more prosthetic articular surfaces, wherein the prosthesis comprises a contoured plate having two sides, one side of which is a bone-contacting side and the other side of which is an articular side, and at least one post extending from the bone contacting side.

18. The surgical kit of claim 17, wherein the cross-sectional geometry of the prosthesis is selected from the group of geometries consisting of a circular geometry, an oval geometry, a └-shaped geometry, a ┘-shaped geometry, and a triangular geometry.

19. The surgical kit of claim 17, further comprising at least one prosthesis from each of the following categories of cross-sectional geometries: circular, oval, └-shaped, ┘-shaped, and triangular.

20. The surgical kit of claim 19, wherein each of the prosthesis for each category of cross-sectional geometries has a different radius of curvature.

21. The surgical kit of claim 19, wherein each of the prosthesis for each category of cross-sectional geometries has a different size.

22. The surgical kit of claim 17, further comprising a reamer bit for reaming out a volume of cartilage and subchondral bone to accept the prosthetic articular surface.

23. The surgical kit of claim 17, further comprising a drill bit for drilling a hole in the bone to accept the post of the prosthetic articular surface.

24. The surgical kit of claim 17, further comprising guide tools corresponding to the cross-sectional geometry, size, and radius of curvature of each of the prostheses in the surgical kit.

25. The surgical kit of claim 24, wherein the guide tool comprises a template with a shape and size substantially similar to the cross-sectional geometry and size of a given prosthesis, and a top surface with a radius of curvature substantially similar to the radius of curvature of the given prosthesis.

26. The surgical kit of claim 17, further comprising:
a reamer bit;
a guide tool comprising a template with a shape and size substantially similar to the cross-sectional geometry and size of a prosthetic articular surface, and a top surface with a radius of curvature substantially similar to the radius of curvature of the given prosthesis; and
a depth gauge capable of engaging the reamer bit and the guide tool.

27. The surgical kit of claim 26, wherein the depth gauge comprises:
a collar capable of releasably engaging the reamer bit and slidably disposable on an inner surface of the guide tool; and
a sleeve connected to the collar and capable of contacting a top surface of the guide tool, wherein the sleeve is not capable of fitting within the confines of the guide tool.

28. A guide tool for guiding a reamer bit during removal of bone for implantation of a prosthetic articular surface, comprising a template with a shape and size substantially similar to the cross-sectional geometry and size of the prosthetic articular surface, and a top surface with a radius of curvature substantially similar to the radius of curvature of the prosthetic articular surface.

29. A prosthetic articular surface for implantation in a knee, comprising a contoured plate having two sides, one side of which is a bone-contacting side and the other side of which is an articular side, and at least one post extending from the bone contacting side; wherein the cross-section of the contoured plate is selected from a circle, oval, └-shape, ┘-shape, and triangular shape.
